(19) United States
(12) Patent Application Publication
(10) Pub. No.: US 2021/0379278 A1
(43) Pub. Date: Dec. 9, 2021

(54) SYRINGE HOLDER

(71) Applicant: Merck Sharp & Dohme Corp., Rahway, NJ (US)

(72) Inventors: Shane Owen Peechatka, Blue Bell, PA (US); Kristen Mancini, Hatfield, PA (US)

(73) Assignee: Merck Sharp & Dohme Corp., Rahway, NJ (US)

(21) Appl. No.: 17/336,464

(22) Filed: Jun. 2, 2021

Related U.S. Application Data
(60) Provisional application No. 63/035,011, filed on Jun. 5, 2020.

(51) Int. Cl. A61M 5/00; A61M 5/31; B33Y 80/00

(52) U.S. Cl. CPC A61M 5/008; B33Y 80/00; A61M 5/3137

(57) ABSTRACT

A syringe holder for holding and stabilizing syringes, the syringe holder comprising a base, and both a vertical mount and a horizontal mount extending from one side of the base. The vertical mount comprises a cylindrical cavity configured to receive and rotatably attach the syringe to the base such that the syringe is held to the base in a generally vertical orientation, while the horizontal mount comprises a lower barrel support and an upper barrel support configured to receive and rotatably attach the bottom end of the barrel and the top end of the barrel of the syringe to the base such that the syringe is held to the base in a generally horizontal orientation. In both orientations, the syringe holder of the present invention provides easy access to most of the exterior surfaces of the syringes and reduces safety hazards associated with creating sample defects on syringes for purposes of qualification testing for syringe inspectors.

### FIELD OF ART

The present invention relates generally to holders, stands and clamps for pharmaceutical product containers, and more particularly to holders, stands and clamps for syringes.

### RELATED ART

Pharmaceutical products, such as drugs and vaccines, are often stored, shipped and/or distributed in syringes made from glass or plastic. To ensure that these syringes are free of potentially serious defects, such as breaks, cracks, chips, cuts, dents, nicks, scrapes, or abrasions on the inside or outside surfaces of the syringes when they reach consumers, and to ensure that the solutions inside the syringes do not contain any dangerous or otherwise undesirable particulates, drug and vaccine manufacturers frequently employ skilled inspectors to inspect batches of drugs and vaccines, or to monitor drug and vaccine production lines, to identify and remove syringes containing such defects.

The inspectors that are employed to identify and remove defective syringes from drug and vaccine batches or production lines typically go through training, testing and qualification program designed to confirm that they have achieved (or maintained) a certain level of proficiency at doing the job. In certain cases, the training, testing and qualification programs involve intentionally creating defective syringes, mixing those defective syringes among non-defective syringes in a larger batch or production line, and then determining through testing how well each inspector or inspector candidate performs the task of identifying and removing those defective syringes from the batch or production line. Often, candidates who wish to be certified as inspectors, as well as previously qualified inspectors seeking to be re-certified, must identify and remove a certain number of defective syringes from a batch or production line before they are certified (or re-certified) to carry out inspections (or continue carrying out inspections) on real, commercial batches or production lines for drugs or vaccines.

For testing purposes, testers alter non-defective syringes to change them into damaged and defective syringes with the help of a variety of handheld instruments and tools, such as, for example, knives, needles, nails, files, razor blades, spikes, etching devices or hacksaws. Testers also use a variety of handheld power tools, such as electric dremels, drills, rotary saws or augers, to create damaged and defective syringes. Sometimes, testers create damaged and/or defective syringes using heat- and flame-producing devices, such as soldering irons, cigarette lighters or handheld torches. Sometimes needles are used to dent or puncture syringes and/or introduce particulates into the solutions contained in the syringes. All of these handheld instruments and tools have the potential to cause serious injury to humans while they are being used to create a damaged or defective syringe, especially if the syringe is not held securely in a stabile position during the defect creation. While creating sample defects, testers frequently try to hold and manipulate the syringe with one hand while holding and manipulating the defect-creating handheld instrument or tool in the other hand. Holding and manipulating the syringe in one hand typically brings that hand into close proximity with the business ends of a variety of very sharp, very pointed, very cold and/or very hot tools. This creates a serious safety hazard for testers.

Prior art syringe holders have proven to be unsuitable for holding syringes securely and safely while performing tasks associated with creating sample defects for inspector qualification tests. For example, many prior syringe holders are designed to hold a collection of multiple syringes arranged in close proximity to each other and in a substantially uniform line or grid pattern to facilitate filling multiple syringes simultaneously, either by hand or by operation of a machine. The construction of such prior art syringe holders does not permit easy access to most of the exterior surfaces of a single syringe.

Prior to the development of the present invention, there was no syringe-holding device available that could adequately hold the syringes stable and securely in either a vertical or a horizontal position during defect creation so that testers would not need to use one of their hands to hold the syringe steady while the instrument or tool being used to create the defect is in contact with the syringe. There was also no syringe-holding device suitably configured to permit 360-degree access to that portion of the syringe currently being worked on to create a defect (i.e., easy access to the full circumference of the barrel of the syringe).

Accordingly, there is still a need in the pharmaceutical and pharmaceutical packaging fields for a syringe holder configured to hold and stabilize a syringe in either a vertical or a horizontal position, while exposing and providing easy access to different portions of the syringe.

### SUMMARY OF THE INVENTION

As will be described in more detail below, aspects and embodiments of the present invention address the above-described needs, as well as other deficiencies and problems associated with known syringe-holding devices, by providing a syringe holder for a syringe comprising a base, a vertical mount and a horizontal mount. The base has a top side and a bottom side. Both of the vertical and horizontal mounts extend from the top side of the base. The vertical mount is configured to receive and rotatably secure the syringe to the base in a generally vertical orientation (i.e., so that the barrel of the syringe lies in a plane that is more or less perpendicular to the plane of the top side of the base), while the horizontal mount is configured to receive and rotatably secure the syringe to the base in a generally horizontal orientation (i.e., so that the barrel of the syringe lies in a plane that is more or less parallel with the plane of the top side of the base). In either orientation, or both of them, the syringe holder of the present invention provides easy access to most of the exterior surfaces of the syringes.

Typically, syringes used for drugs and vaccines have a movable piston extending through the inside a barrel (sometimes referred to as a tube), and a discharge orifice and a shoulder located at or near the top end of the barrel. At the opposite (or bottom) end of the barrel, there are usually one or more finger flanges attached to the circumference of the bottom end of the barrel, a piston rod attached to the bottom of the piston, and a thumb blade designed to facilitate pushing the piston rod (and therefore the piston) deeper into the barrel. The outside of the barrel may or may not include scale markings to indicate the volume of solution in the barrel. Accordingly, embodiments of the syringe holder of the present invention provide support for syringes having these physical characteristics. For example, the vertical mount, which extends from the top side of the base, comprises a cylindrical wall defining a cylindrical cavity extending through the base with a first opening on the top side of the base and a second opening on the bottom side of the base, wherein the cylindrical cavity, the first opening and the second opening are adapted to receive and hold (i) the bottom end of the barrel, and (ii) the top end of the barrel. In other words, the cylindrical cavity of the vertical mount of the syringe holder is advantageously configured (in terms of its geometry) so that it can receive and hold both the top end of the barrel and the bottom end of the barrel, although not simultaneously. Thus, the same vertical mount can sometimes be used to hold the top end of the barrel, while at other times it can be used to hold the bottom end of the barrel. Whether the vertical mount is used to hold the top end of the barrel or the bottom end of the barrel will be up to the user, depending on which end of the barrel the user wants to expose for purposes of creating defects. When the bottom end of the barrel is inserted into the vertical mount of the syringe holder so that the syringe is in an upright position, the top end of the barrel, the shoulder and the discharge orifice of the syringe are exposed and substantially unobstructed by the vertical mount, thereby permitting easy access to these portions of the syringe to facilitate creating sample defects thereon. When the syringe is turned upside down and the top end of the barrel is inserted into the same vertical mount of the syringe holder, potions of the bottom end of the barrel, the finger flange(s), a portion of the piston rod and the thumb blade of the syringe are exposed and generally unobstructed by the vertical mount, thereby permitting easy access to these portions of the syringe so as to facilitate creating sample defects thereon.

In some embodiments, at least part of the cylindrical wall defining the cylindrical cavity extends upward from the top side of the base so that one of the two openings in the cylindrical cavity lies in a plane located above the top side of the base. At least part of the cylindrical wall defining the cylindrical cavity also extends downward and through the bottom side of the base so that the second opening in the cylindrical cavity is coincides with the bottom side of the base, which creates a hole in the bottom side of the base. The syringe may be inserted into the vertical mount of the base in two different ways. The first way of inserting the syringe into the vertical mount of the syringe holder is by pushing the discharge orifice, shoulder and top end of the barrel on the syringe down through the first opening of the vertical mount on the top side of the base, which leaves the bottom end of the barrel, the finger flange(s) and the thumb blade pointing up and substantially unobstructed by the vertical mount. the discharge orifice, the shoulder and/or the top end of the barrel may (or may not) extend all the way through and out of the bottom side of the base. The second way of inserting the syringe into the vertical mount of the syringe holder is by pushing the discharge orifice, the shoulder and the top end of the barrel on the syringe up through the second opening on the bottom side of the base until the finger flange(s) on the barrel prevent the barrel from moving completely through the base. In this situation, the middle portion of the barrel, the top end of the barrel, the shoulder and the discharge orifice of the syringe will all be exposed above the first opening on vertical mount on the top side of the base, and substantially unobstructed by the vertical mount of the syringe holder. Preferably, the bottom side of the base includes one or more depressions configured to permit the finger flange(s) and/or the thumb blade of the syringe to move into a position that lies above the plane of the bottom side of the base, thereby permitting the remaining portions of the bottom side of the base to lie flush with surface of a table or desk while the syringe is being held by the vertical mount of the syringe holder.

The horizontal mount, which also extends from the top side of the base, is configured to receive and rotatably secure the syringe to the base in a generally horizontal orientation (i.e., so that the central axis of the barrel of the syringe lies in a plane that is roughly parallel to the plane of the top side of the base). In this orientation, both the top end of the barrel and the bottom end of the barrel of the syringe, as well as certain portions of the discharge orifice, shoulder, finger flange(s) and thumb blade, are exposed and substantially unobstructed by the horizontal mount itself. In this orientation, a portion of the piston rod may also be exposed, depending on whether the piston rod is in an extracted or a retracted position in the barrel. The horizontal mount comprises a lower barrel support for the bottom end of the barrel of the syringe and an upper barrel support for the top end of the barrel of the syringe. The lower barrel support comprises a first inverted rounded arch defining a first rounded slot adapted to receive and support the bottom end of the barrel (closer to the finger flange(s)) of the syringe, and the upper barrel support comprises a second inverted rounded arch defining a second rounded slot adapted to receive and support the top end of the barrel of the syringe (closer to the shoulder and discharge orifice).

Preferably, the lower barrel support and the upper barrel support of the horizontal mount are separated from the plane of the top side of the base so that there is at least some degree of spacing (or clearance) underneath the syringe, i.e., between the top side of the base and the downward facing portions of the syringe, when the syringe is resting on the lower barrel support and the upper barrel support of the horizontal mount. This spacing promotes and facilitates easier access to a greater amount of the surface area of the syringe with hand tools and power tools for creating defects, and also make it easier to rotate the syringe while it is resting on the horizontal mount. To achieve the desired spacing, some embodiments of the syringe holder suitably include a substantially rigid lower barrel support riser disposed between the first inverted rounded arch of the lower barrel support and the top side of base, as well as a substantially rigid upper barrel support riser disposed between the second inverted rounded arch of the lower barrel support and the top side of base, thereby separating the first and second inverted rounded arches from the top side of the base. The first and second inverted rounded arches are curved to match the curvature of the barrel.

Typically, it will be necessary or desirable to secure the syringe holder of the present invention to a table or work bench so that the device will not move while it is being used to create defects in syringes. Accordingly, some embodiments of the present invention further include one or more recesses (or “cut outs”) in at least one edge of the top side of the base, which define one or more niches, respectively, that are configured to receive and accept the jaws of one or more table clamps in such a way that the tops of the table clamp jaws do not rise significantly above the top side of the base while the table clamp is holding the base tight to the table. With this configuration, the tops of the table clamp jaws are much less likely to interfere with easy access to the syringes currently being held and supported by the vertical or horizontal mounts on the syringe holder.

Preferably, but not necessarily, the vertical and horizontal mounts are designed to hold the same syringe (or at least syringes of the same dimensions) in both of the available (vertical and horizontal) orientations. Accordingly, the diameter (D1) of the cylindrical cavity in the vertical mount may be equal in size to the diameter (D2) of the first inverted rounded arch of the lower barrel support of the horizontal mount. In other embodiments, however, the diameter (D1) of the cylindrical cavity in the vertical mount may be different from the diameter (D2) of the first inverted rounded arch of the lower barrel support. For example, the diameter (D2), or the radius, of the first inverted rounded arch in the horizontal support may be considerably smaller or considerably larger than the diameter (or radius) of the cylindrical cavity in the vertical mount without departing from the scope of the claimed invention.

In some embodiments, the cylindrical cavity in the vertical mount may have a diameter D1 that is slightly smaller than the diameters of the bottom end and/or the top end of the barrel of the syringe so that the bottom end of the barrel and/or the top end of the barrel may be removably “snap-fitted” into place in the cylindrical cavity so that the syringe is more securely held in place in a vertical orientation. Similarly, the first rounded slot in the lower barrel support of the horizontal mount may have a diameter (D2) that is slightly smaller than the diameter of the bottom end of the barrel of the syringe so that the bottom end of the barrel of the syringe may be removably “snap-fitted” into the first rounded slot of the lower barrel support of the horizontal mount. The second rounded slot in the upper barrel support of the horizontal mount may have a diameter (D3) that is slightly smaller than the diameter of the top end of the barrel of the syringe so that the top end of the barrel of the syringe may be removably snap-fitted into the second rounded slot of the upper barrel support. As shown in the figures and described in more detail below, the second inverted rounded arch of the upper barrel support of the horizontal mount may be elongated to define an elongated trough configured to support the top end of the barrel of the syringe when the syringe is placed in said horizontal orientation on the horizontal mount.

Holding the syringe in a generally vertical orientation means the syringe may be held by the syringe holder in any orientation in which the angle between the major axis of the barrel of the syringe and the plane of the top side of the base is greater than or equal to 45 degrees (and less than or equal to 135 degrees). Holding the syringe in a generally horizontal orientation means the syringe may be held by the syringe holder in any orientation in which the angle between the major axis of the syringe and the plane of the top side of the base is less than or equal to 45 degrees (or greater than or equal to 135 degrees). Typically, however, the syringe holder will be configured to hold the syringe at an angle of about 90 degrees (roughly perpendicular) relative to the plane of the top side of the base, or at an angle of about 0 (zero) degrees relative to the plane of the top side of the base, or both 90 degrees and 0 (zero) degrees (parallel) relative to the top side of the base, because these angles typically provide the greatest access to the most surface area of the syringe being held without having to rotate the syringe in the holder.

The syringe holder of the present invention may be constructed by separately fabricating the component parts, such as the base, the vertical mount and the horizontal mount, and then joining the component parts together to form the completed syringe holder. Alternatively, syringe holders configured in accordance with embodiments of the present invention may be constructed from a single piece of construction material (unitary construction), such as by using a three-dimensional (3D) printer or by injection molding. For purposes of fabricating the syringe holder of the present invention by 3D printing, the material used for the 3D printing of the syringe holder may include, for example, polyamide thermoplastic (Nylon), polyetherimide (PEI) thermoplastic, polyether ether keytone (PEEK) thermoplastic, or Acrylonitrile Butadiene Styrene (ABS) thermoplastic. However, it will be recognized by those skilled in the art that one or more other materials may be suitably adapted and used to fabricate syringe holders of the present invention.

### BRIEF DESCRIPTION OF THE DRAWINGS

The present invention and various aspects, features and advantages thereof are explained in detail below with reference to exemplary and therefore non-limiting embodiments, and with the aid of the drawings, which constitute a part of this specification and include depictions of the exemplary embodiments. In these drawings:

FIG. 1 shows a front and right perspective view of a syringe holder according to one embodiment of the present invention.

FIGS. 2A, 2B, 2C and 2D show, respectively, a top view, a top and front perspective view, a top and left perspective view, and a top and right perspective view of the syringe holder according to one embodiment of the present invention.

FIGS. 3A and 3B show, respectively, a top and rear perspective view, and a bottom view of the syringe holder according to an embodiment of the present invention.

FIGS. 4A, 4B and 4C show, respectively, a front elevation view, a rear elevation view, and a right elevation view of the syringe holder according to an embodiment of the present invention.

FIGS. 5A and 5B show, respectively, representative diagrams of an exemplary syringe that could be held and stabilized by embodiments of the present invention. FIG. 5A shows the syringe in a generally vertical orientation and FIG. 5B shows the syringe in a generally horizontal orientation.

FIG. 6A shows a close-up view of the vertical mount as it would appear from above the vertical amount.

FIG. 6B shows a cross sectional view of the vertical mount.

FIGS. 7A, 7B and 7C show, respectively, a close-up view of the horizontal mount as viewed from above the horizontal mount, a cross-sectional view of the lower barrel support of the horizontal mount, and a cross-sectional view of the upper barrel support of the horizontal mount.

FIG. 8 shows a left and top perspective view of another embodiment of the present invention, wherein the locations of the vertical and horizontal mounts are swapped relative to the examples of the invention depicted in the previously-described figures.

### DETAILED DESCRIPTION OF EXEMPLARY EMBODIMENTS

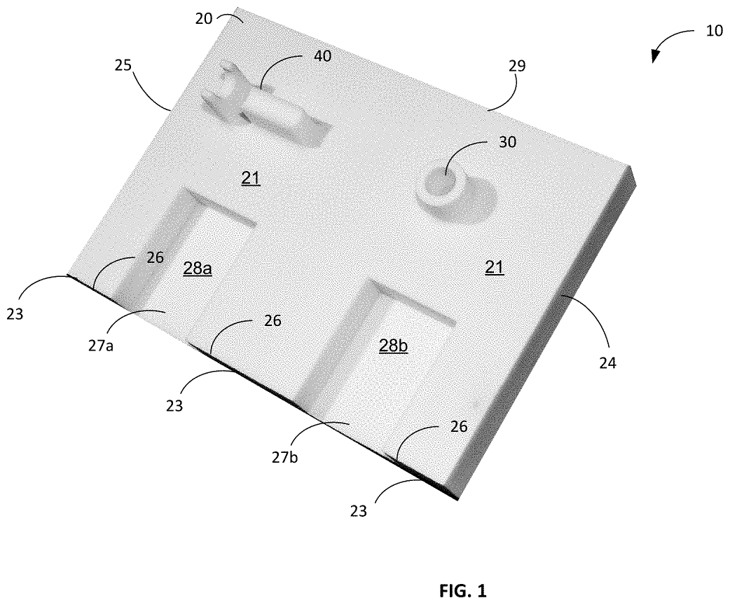

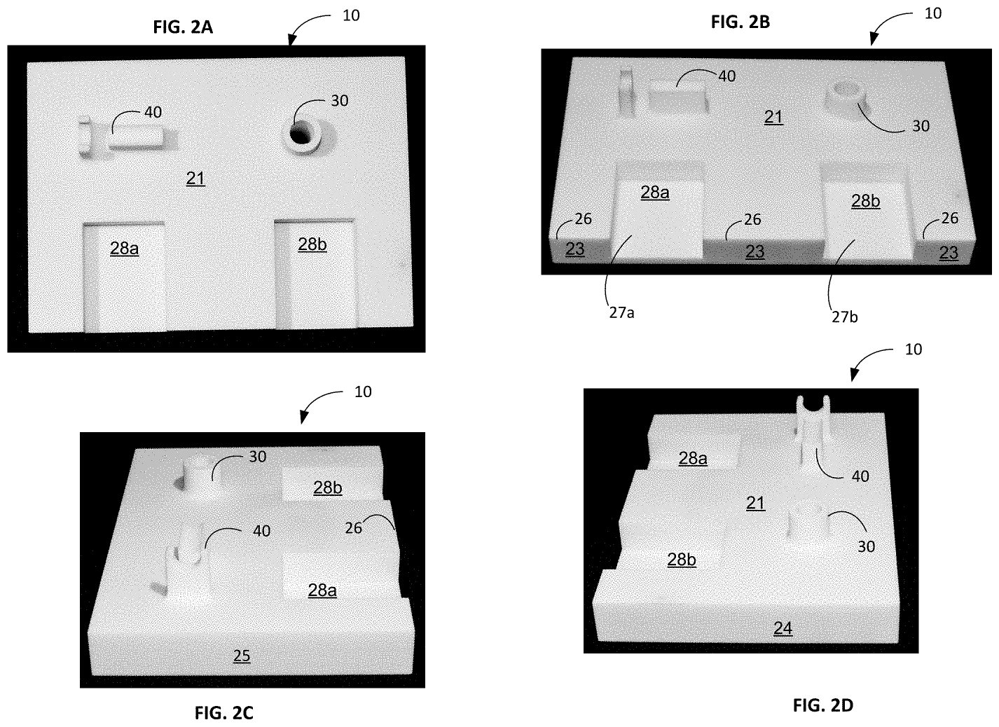

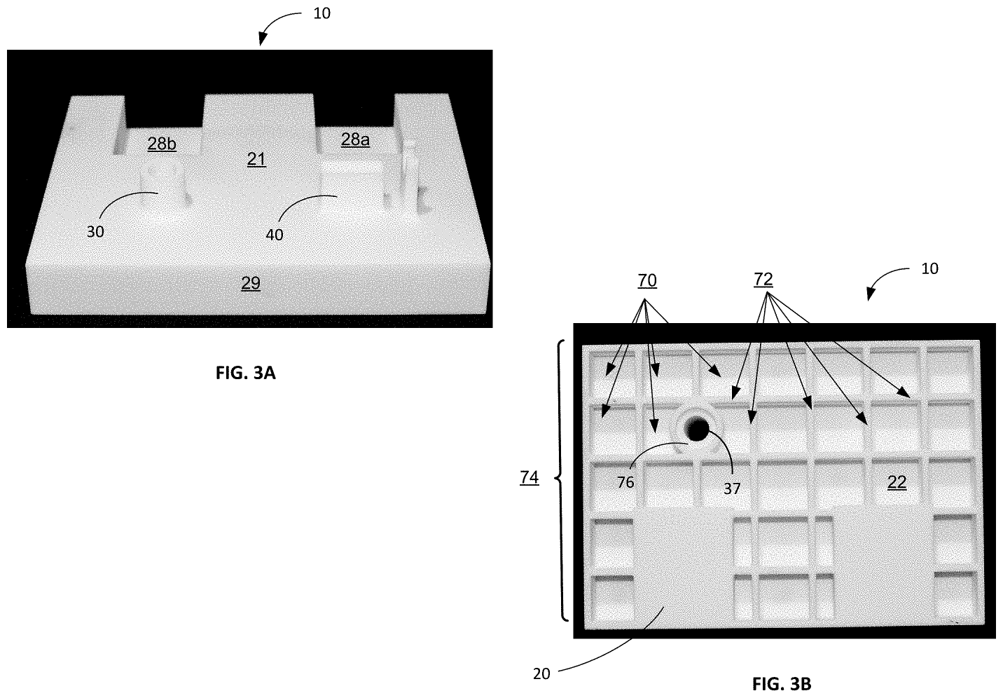

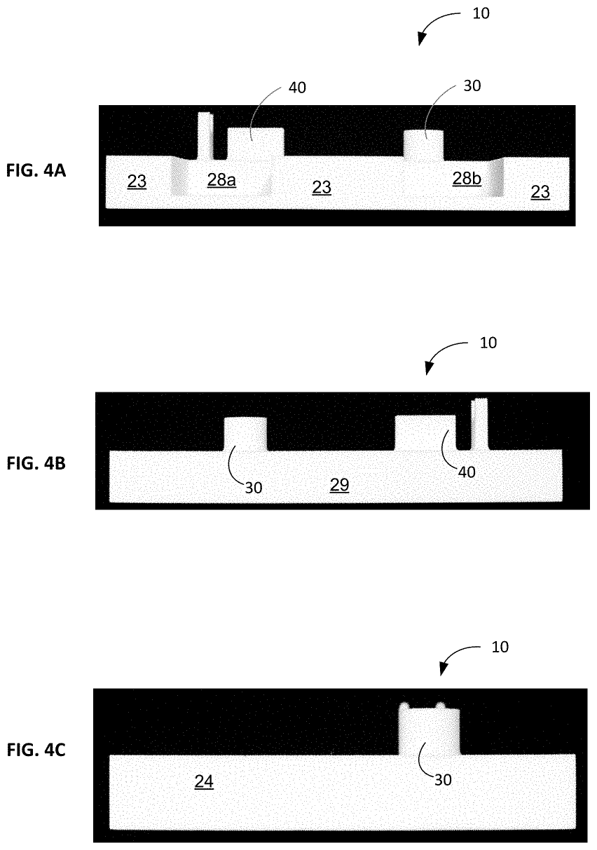

Exemplary syringe holders according to embodiments of the present invention will now be described in more detail with reference to the figures. The syringe holder of the present invention is generally designated as 10 in the accompanying figures. FIG. 1 shows a front and right perspective view of a syringe holder 10 according to one embodiment of the present invention. FIGS. 2A, 2B, 2C and 2D show, respectively, a top view, a top and front perspective view, a top and left perspective view, and a top and right perspective view of the syringe holder 10. FIGS. 3A shows a top and rear perspective view of the syringe holder 10. FIG. 3B shows a bottom view of the syringe holder 10. FIGS. 4A, 4B and 4C show, respectively, a front elevation view, a rear elevation view, and a right elevation view of the syringe holder 10.

As illustrated in the figures, the syringe holder 10 comprises a base 20 having a top side 21, a bottom side 22, a front side 23, a right side 24, a left side 25 and a rear side 29. The front side 23, right side 24, left side 25 and rear side 29 are configured to form substantially vertical walls that are relatively perpendicular to the top side 21 and the bottom side 22 of the base 20. While the syringe holder 10 depicted in the figures comprises a substantially rectangular solid that is substantially rectilinear in form, it will be recognized and appreciated by those skilled in the art that syringe holders of the present invention may alternatively be constructed to form a variety of different geometrical shapes, including for example, a planar disk, triangle, pentagon, hexagon, octagon, or the like. In some embodiments, the surface of the bottom side 22 is substantially planar in shape (e.g., flat). In other embodiments, the surface of the bottom side 22 may comprise one or more voids or cutouts (or a multiplicity of voids or cutouts) to reduce the amount of material in the syringe holder 10, and thereby reduce the weight of the syringe holder 10. FIG. 3B shows such an embodiment, wherein the bottom side 22 comprises a multiplicity of rectangular cutouts 70 and intersecting walls 72 defining a grid-like pattern 74 on the surface of the bottom side 22.

In still other embodiments, the top side 21 and/or the bottom side 22 of the base 20 may not be substantially planar or flat. For example, the top side 21 and/or the bottom side 22 of the base 20 could have a shape or geometry that is concave, convex, inclined, elliptical, reticulated, annular, beveled or grooved, depending, for example, on the shapes of the syringes to be fastened to the holder or the tools and/or machines expected to be used with the syringe holder 10 to create defects.

Cut into the edge 26 of the base 20, where the top side 21 and the front side 23 of the base 20 intersect, there is provided a pair of recesses 27a and 27b (or “cut outs”) defining a pair of niches 28a and 28b , respectively, which are configured to receive the jaws of a pair of clamps, such as a pair of table clamps or a pair of “c-clamps” (not shown in the figures), which can be used to fasten the base 20 of the syringe holder 10 securely to a table or workbench (also not shown in the figures). Preferably, the niches 28a and 28b are sufficiently deep, relative to the jaws of the clamps and the plane of the top side 21 of the base 20, so that the jaws of the clamp do not extend above the plane of the top side 21 of the base 20, and thereby are able to fasten and hold the syringe holder 10 securely to a desk, table or workbench without interfering with, or reducing easy access to the syringe or syringes mounted in the vertical mount 30 and/or the horizontal mount 40 of the syringe holder 10, and do not pose a safety hazard due to the nearby operation and manipulation of hand or power tools being used to create sample defects in the syringes.

The pair of recesses 27a and 27b , as well as the pair of niches 28a and 28b , also may be located on other edges of the base 20, such as the edge defining the intersection of the top side 21 and the rear side 29 of the base 20, the edge defining the intersection of the top side 21 and the right side 24 of the base 20, or the edge defining the intersection of the top side 21 and the left side 25 of the base 20. Depending on the size and shape of the syringe holder 10, as well as the geometry of the table or workbench to which the syringe holder 10 will be attached, the syringe holder 10 may have only one recess, as well as three or more recesses, without departing from the scope of the claimed invention.

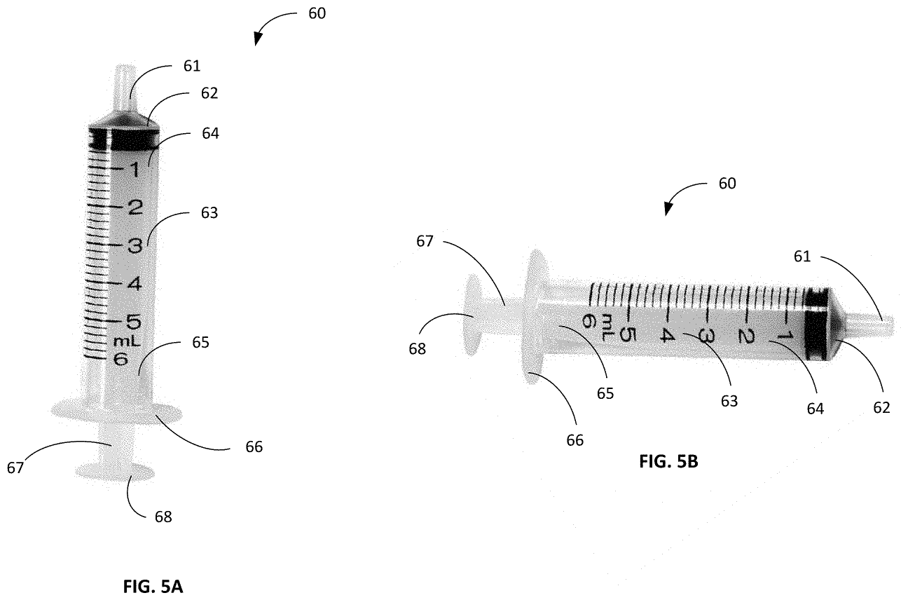

As stated above, the syringe holder 10 of the present invention also includes a vertical mount (designated generally as 30 in the figures) and a horizontal mount (designated generally as 40 in the figures), both of which are configured to extend upward from the top side 21 of the base 20 of the syringe holder 10. To simplify the discussion and improve the comprehension of the following detailed description of the geometries and the functions of the component parts of the vertical mount 30 and the horizontal mount 40, FIGS. 5A and 5B show diagrams of an exemplary syringe 60. FIG. 5A shows the syringe 60 in a generally vertical orientation (as the syringe 60 would be oriented while it is attached to the vertical mount 30), and FIG. 5B shows the syringe 60 in a generally horizontal orientation (as the syringe 60 would be oriented while it is attached to the horizontal mount 40). As FIGS. 5A and 5B illustrate, the exemplary syringe 60 has a discharge orifice 61, a shoulder 62, a barrel 63, a top end of the barrel 64, a bottom end of the barrel 65, a finger flange 66, piston rod 67 and a thumb blade 68. The discharge orifice 61 may (or may not) be attached to a hypodermic needle or a nozzle (not shown in FIGS. 5A and 5B).

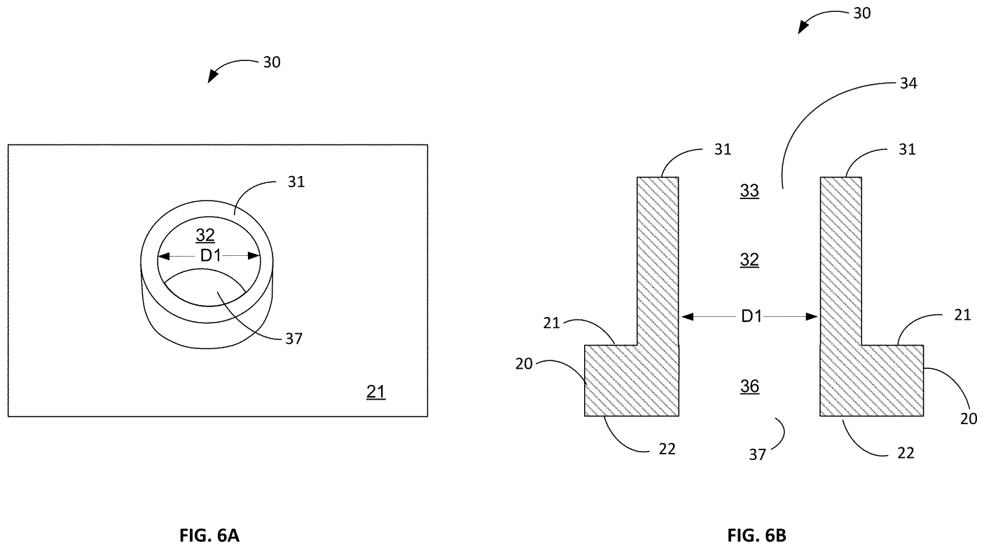

Turning now to the detailed descriptions of the vertical mount 30 of the syringe holder 10, FIG. 6A shows a close-up view of the vertical mount 30 as it would appear if viewed from above the vertical mount 30. FIG. 6B shows a cross-sectional view of the vertical mount 30 as viewed from the front side 23 of the syringe holder 10. As illustrated in FIGS. 6A and 6B, the vertical mount 30 comprises a cylindrical wall 31 defining a cylindrical cavity 32 that extends all the way through the base 20. The cylindrical cavity 32 has two openings 33 and 37 located, respectively, at the top end 34 and at the bottom end 36 of the cylindrical cavity 32. The openings 33 and 37 are adapted to receive and rotatably hold the discharge orifice 61, the shoulder 62, the barrel 63, and/or the top end 64 of the barrel 63 of the syringe 60. The cylindrical cavity 32 and the openings 33 and 37 at the top end 34 and the bottom end 36 of the cylindrical cavity 32 may also be configured to receive and rotatably hold the bottom end 65 of the barrel 63, the finger flange 66, the piston rod 67 and/or the thumb blade 68.

When the vertical mount 30 of the syringe holder 10 is used to hold the bottom end 65 of the barrel 63, the finger flange 66, the piston rod 67 and/or the thumb blade 68 of the syringe 60 so that the syringe 60 is in an upright position (as shown in FIG. 5A), the discharge orifice 61, the shoulder 62, the barrel 63, and/or the top end 64 of the barrel 63 of the syringe 60 are exposed and remain generally unobstructed by the vertical mount 30. This enables easy access to these portions of the syringe 60 to facilitate creating sample defects thereon. When the vertical mount 30 of the syringe holder 10 is used to hold the discharge orifice 61, the shoulder 62, the barrel 63, and/or the top end 64 of the barrel 63 of the syringe 60 so that the syringe 60 is inverted (i.e., upside down), the bottom end 65 of the barrel 63, the finger flange 66, the piston rod 67 (if it is extended) and/or the thumb blade 68 of the syringe 60 are exposed and generally unobstructed by the vertical mount 30, which facilitates easy access to these portions of the syringe 60 for the purpose of using hand or power tools to create sample defects thereon.

Preferably, the diameter D1 of the cylindrical cavity 32 of the vertical mount 30 is slightly smaller than the diameter of the bottom end 65 of the barrel 63 of the syringe 60 (or at the shoulder 62 and top end 64 of the barrel 63 of the syringe 60) so that the bottom end 65 of the barrel 63 of the syringe 60 (or the shoulder 62, top end 64 of the barrel 63 and shoulder 62 of the syringe 60) may be rotatably and removably “snap-fitted” into place inside the cylindrical cavity 32. Snap-fitting of the syringe 60 inside the cylindrical cavity 32 tends to result in the syringe 60 being more securely held in place in a generally vertical orientation while the syringe 60 is worked on to create sample defects.

In some embodiments, and as shown in the figures, at least part of the cylindrical wall 31 defining the cylindrical cavity 32 extends upward from the top side 21 of the base 20 so that the opening 33 in the cylindrical cavity 32 lies in a plane that is located above the plane of the top side 21 of the base 20. As shown best in FIG. 6B, at least one part of the cylindrical wall 31 defining the cylindrical cavity 32 also extends downward and below the plane of the top side 21 of the base 20, so that the top end 34 of the cylindrical cavity 32 lies in a plane above the plane of the top side 21 of the base 20 and the bottom end 36 of the cylindrical cavity 32 lies in a plane below the plane of the top side 21 of the base 20. In other embodiments, the cylindrical wall 31 of the vertical mount 30 may only extend downward from the plane of the top side 21 of the base 20, so that the opening 33 at the top end 34 of the cylindrical cavity 32 will lie substantially in the same plane as the top side 21 of the base 20.

As shown best in FIGS. 6A and 6B, embodiments of the present invention may include a second opening 37 at the bottom end 36 of the cylindrical cavity 32, wherein the plane of the second opening coincides with the plane of the bottom side 22 of the base 20. In such embodiments, instead of inserting the syringe 60 into the syringe holder 10 by pushing the syringe 60 down into the vertical mount 30 of the syringe holder 10 from the top side 21 of the base 20, the syringe 60 may be inserted into the vertical mount 30 of the syringe holder 10 by pushing the syringe 60 up through the second opening on the bottom side 22 of the base 20. Suitably, the syringe 60 may be inserted into the syringe holder 10 by pushing the syringe 60 (discharge orifice-first) through the second opening 37 at the bottom end 36 of the cylindrical cavity 32 when an upright orientation of the syringe 60 is desired while defect is created, or by pushing the syringe 60 (discharge orifice-first) through the first opening 33 at the top end 34 of the cylindrical cavity 32 when an inverted orientation of the syringe 60 is desired during defect creation.

Typically, but not necessarily, the central axis of the cylindrical cavity 32 defined by the cylindrical wall 31 of the vertical mount 30 will be normal (and/or perpendicular) to the plane of the top side 21 of the base 20. It is understood, however, that the central axis of the cylindrical cavity 32 defined by the cylindrical wall 31 of the vertical mount 30 does not necessarily have to be normal (or perpendicular) to the plane of the top side 32 of the base 20. In some embodiments, for instance, the central axis of the cylindrical cavity 32 defined by the cylindrical wall 31 of the vertical mount 30 may lie at an angle that is anywhere between 45 degrees and 90 degrees relative to the plane of the top side 21 of the base 20, so that the major axis of the syringe 60 held by the vertical mount 30 will also lie at the same angle relative to the plane of the top side 21 of the base 20.

As shown best in FIG. 3B, the bottom side 22 of the base 20 may also include a slot or depression 76 circumscribing the circumference of the second opening 37 at the bottom end 36 of the cylindrical cavity 32 of the vertical mount 30. (The bottom end 36 and the cylindrical cavity 32 of the vertical mount 30 are shown best in the cross-sectional illustration of the vertical mount 30 contained in FIG. 6B). The slot or depression 76 is adapted to receive and hold the finger flange 66 and/or the thumb blade 68 on the syringe 60. Ideally, the slot or depression 76 on the bottom side 22 of the base 20 will permit the finger flange 66 and/or the thumb blade 68 of the syringe 60 to slide up and into a position within the syringe holder 10 so that the finger flange 66 and/or the thumb blade 68 will come to rest above the plane of the bottom side 22 of the base 20, thereby permitting the remaining portions of the bottom side 22 of the base 20 to lie flush with surface of a table or desk while the syringe 60 is being held by the vertical mount 30 of the syringe holder 10.

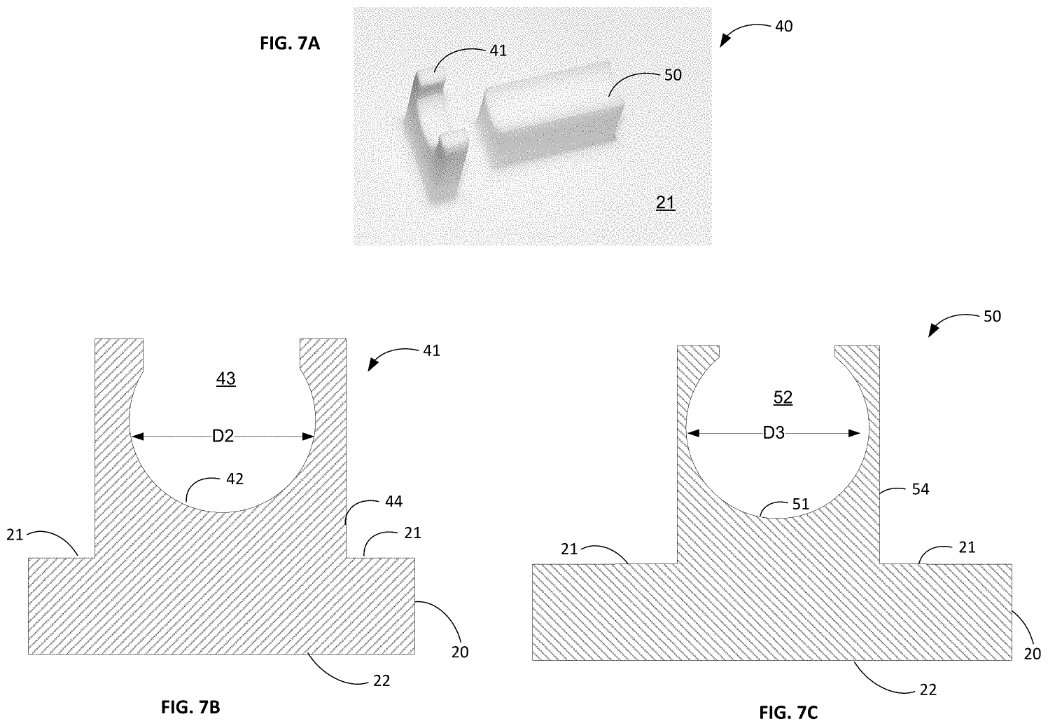

FIG. 7A shows a close-up view of the horizontal mount 40 as it would appear if viewed from above the horizontal mount 40. Like the vertical mount 30, the horizontal mount 40 also extends from the top side 21 of the base 20 and is configured to receive and rotatably secure the syringe 60 to the base 20. However, the horizontal mount 40 secures the syringe 60 in a generally horizontal orientation so that portions of both the upper and the lower ends of the syringe 60 are exposed and generally unobstructed by the horizontal mount 40 itself. As shown in FIG. 7A, the horizontal mount 40 includes two components extending from the top side 21 of the base 20; namely, a lower barrel support 41 to support the bottom end 65 of the barrel 63 of the syringe 60, and an upper barrel support 50 to support the top end 64 of the barrel 63 of the syringe 60. FIG. 7B shows a cross-sectional view of the lower barrel support 41, and FIG. 7C shows a cross-sectional view of the upper barrel support 50 of the horizontal mount 40.

The lower barrel support 41 (shown best in FIG. 7B) of the horizontal mount 40 comprises a first inverted rounded arch 42 defining a first rounded slot 43 adapted to receive and support the lower end 65 of the barrel 63 of the syringe 60 while the syringe 60 is attached to the lower barrel support 41 and the upper barrel support 50 and lying in a generally horizontal orientation. The upper barrel support 50 (shown best in FIG. 7C) comprises a second inverted rounded arch 51 defining a second rounded slot 52 adapted to receive and support a section of the top end 64 of the barrel 63 of the syringe 60 while the syringe 60 is attached to the lower barrel support 41 and the upper barrel support 50 and lying in a generally horizontal orientation.

As illustrated by the examples of the lower barrel support 41 and the upper barrel support 50 shown in FIGS. 7A, 7B and 7C, respectively, the lower barrel support 41 and the upper barrel support 50 of the horizontal mount 40 may be separate components that are spaced apart from each other. The lower barrel support 41 and the upper barrel support 50 of the horizontal mount 40 also may be separated from (i.e., lifted above) the plane of the top side 21 of the base 20 so that there exists at least some measure of spacing (or clearance) underneath the syringe 60, i.e., between the top side 21 of the base 20 and the downward facing portions of the syringe 60, whenever the syringe 60 is resting on the lower barrel support 41 and the upper barrel support 50 of the horizontal mount 40. This spacing promotes and facilitates easier access to a greater portion of the syringe 60 with hand and/or power tools while creating defects, and also make it easier to rotate the syringe 60 while it is resting on the horizontal mount 40.

To achieve the desired spacing (or clearance), the lower barrel support 41 of the horizontal mount 40 may include a substantially rigid lower barrel support riser 44 disposed between the first inverted rounded arch 42 of the lower barrel support 41 and the top side 21 of base 20, thereby putting distance between the first inverted rounded arch 42 and the top side 21 of the base 20. Similarly, the upper barrel support 50 of the horizontal mount 40 may also include a substantially rigid upper barrel support riser 54 disposed between the second inverted rounded arch 51 of the upper barrel support 50 and the top side 21 of base 20, thereby putting some distance between the second inverted rounded arch 51 and the top side 21 of the base 20.

Normally, although not necessarily, the diameter D2 of the first rounded slot 43 of the lower barrel support 41 is roughly equal in size to the diameter D1 of the cylindrical cavity 32 of the vertical mount 30 so that the vertical mount 30 and the horizontal mount 40 will easily be able to accommodate moving syringes from the vertical mount 30 to the horizontal mount 40, and vice versa. However, in some embodiments, depending on the geometry of the particular syringe to be held, the diameter D2 of the first rounded slot 43 of the lower barrel support 41 may be substantially different in size compared to the diameter D1 of the cylindrical cavity 32 of the vertical mount 30. Preferably, the diameter D2 of the lower barrel support 41 of the horizontal mount 40 is slightly smaller in size than the diameter of the barrel 63 of the syringe 60 so that the bottom end 65 of the barrel 63 of the syringe 60 may be removably snapped into the first rounded slot 43 defined by the first inverted rounded arch 42 of the lower barrel support 41. Likewise, the diameter D3 of the second rounded slot 52 of the upper barrel support 50 of the horizontal mount 40 is preferably slightly smaller in size than the diameter of the top end 64 of the barrel 63 of the syringe 60, so that the top end 64 of the barrel 63 of the syringe 60 can be removably snapped into place in the second rounded slot 52 of the upper barrel support 50.

Typically, but not necessarily, the central axes of the first rounded slot 43 and the second rounded slot 52 of the lower barrel support 41 and the upper barrel support 51 of the horizontal mount 40 are parallel to the plane of the top side 21 of the base 20. It is understood, however, that the central axes of the first rounded slot 43 and the second rounded slot 52 of the lower barrel support 41 and the upper barrel support 51 of the horizontal mount 40 do not necessarily have to be exactly parallel to the plane of the top side 21 of the base 20. In some embodiments, for instance, the central axes of the first rounded slot 43 and the second rounded slot 52 of the horizontal mount 40 may lie at an angle that is anywhere between 0 degrees and 45 degrees relative to the plane of the top side 21 of the base 20, so that the major axis of the syringe 60 held by the horizontal mount 40 will also lie at the same angle relative to the plane of the top side 21 of the base 20.

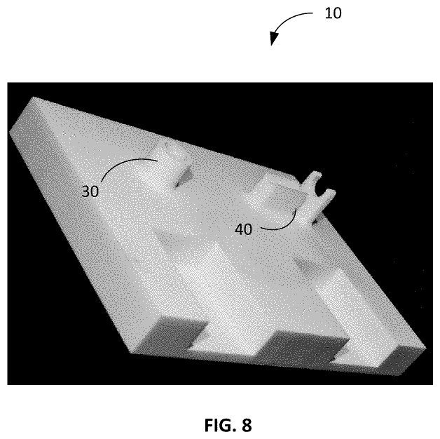

FIG. 8 shows a left-side and top perspective view of a syringe holder 10 according to another embodiment of the present invention, wherein the locations of the vertical mount 30 and the horizontal mount 40 are swapped relative to the examples of the invention depicted in the previously-described figures. It is understood that certain embodiments of the present invention may comprise a plurality of vertical and horizontal mounts so as to permit the syringe holder 10 to hold a plurality of syringes simultaneously.

It is anticipated that embodiments of the invention may be utilized in a variety of fields, including without limitation the fields of manufacturing, storing and distributing pharmaceuticals and containers for pharmaceuticals. Thus, embodiments of the present invention may be used to hold and stabilize syringes while creating sample defects for inspector qualification testing. Under these circumstances, it may be necessary or desirable to construct the syringe holder 10 from a single piece of material (unitary construction) that is easy to clean and/or sanitize, non-flammable, non-combustible, rust-proof, and able to withstand hot, cold and/or wet environments with little or no damage. Accordingly, employing a three-dimensional (3D) printer or an injection molding device to manufacture syringe holders configured in accordance with embodiments of the present invention may be a cost-effective and beneficial approach. For purposes of fabricating the syringe holder of the present invention by 3D printing, the material used for the 3D printing of the syringe holder may include, for example, polyamide thermoplastic (Nylon), polyetherimide (PEI) thermoplastic, polyether ether keytone (PEEK) thermoplastic, or Acrylonitrile Butadiene Styrene (ABS) thermoplastic.

Although the exemplary embodiments, uses and advantages of the invention have been disclosed above with a certain degree of particularity, it will be apparent to those skilled in the art upon consideration of this specification and practice of the invention as disclosed herein that alterations and modifications can be made without departing from the spirit or the scope of the invention, which are intended to be limited only by the following claims and equivalents thereof

What is claimed is:

1. A syringe holder for a syringe, the syringe having a barrel with a top end and a bottom end, a piston inside the barrel, a discharge orifice and a shoulder located at or near the top end of the barrel, one or more finger flanges attached to the bottom end of the barrel, a piston rod attached to the bottom of the piston and extending from the bottom of the barrel, and a thumb blade attached to the piston, the syringe holder comprising:
a) a base having a top side and a bottom side;
b) a vertical mount, disposed on the top side of the base, adapted to receive and rotatably attach the syringe to the base in a generally vertical orientation, the vertical mount comprising a cylindrical wall defining a cylindrical cavity extending through the base with a first opening on the top side of the base and a second opening on the bottom side of the base, the cylindrical cavity, the first opening and the second opening being adapted to receive and hold (i) the bottom end of the barrel so that the top end of the barrel, the shoulder and the discharge orifice of the syringe are exposed and substantially unobstructed by the vertical mount, and (ii) the top end of the barrel, the shoulder and the discharge orifice so that the bottom end of the barrel, the one or more finger flanges and the thumb blade of the syringe are exposed and substantially unobstructed by the vertical mount;
c) a horizontal mount, disposed on the top side of the base, adapted to receive and rotatably attach the syringe to the base in a generally horizontal orientation so that at least portions of the top end of the barrel, the bottom end of the barrel, the finger flanges, the thumb blade, the shoulder and the discharge orifice of the syringe are exposed simultaneously and substantially unobstructed by the horizontal mount; and
d) wherein the horizontal mount comprises a lower barrel support for the bottom end of the barrel and an upper barrel support for the top end of the barrel, the lower barrel support comprising a first inverted rounded arch defining a first rounded slot adapted to receive and hold the bottom end the barrel, and the upper barrel support comprising a second inverted rounded arch defining a second rounded slot adapted to receive and hold the top end of the barrel.

2. The syringe holder of claim 1, wherein:
a) at least part of the cylindrical wall defining the cylindrical cavity extends from the top side of the base; and
b) the first opening in the cylindrical cavity lies in a plane located above the top side of the base.

3. The syringe holder of claim 1, wherein:
a) at least part of the cylindrical wall defining the cylindrical cavity extends below the top side of the base; and
b) the first opening in the cylindrical cavity is substantially coplanar with the top side of the base.

4. The syringe holder of claim 1, wherein:
a) at least one part of the cylindrical wall defining the cylindrical cavity extends above the top side of the base; and
b) at least another part of the cylindrical wall defining the cylindrical cavity extends below the top side of the base.

5. The syringe holder of claim 4, wherein the second opening is substantially coplanar with the bottom side of the base.

6. The syringe holder of claim 1, further comprising:
a) a substantially rigid lower barrel support riser disposed between the first inverted rounded arch of the lower barrel support and the top side of the base; and
b) a substantially rigid upper barrel support riser disposed between the second inverted rounded arch of the upper barrel support and the top side of the base.

7. The syringe holder of claim 1, further comprising a recess in at least one edge of the top side of the base, the recess defining a niche configured to receive a jaw of a table clamp.

8. The syringe holder of claim 1, further comprising a second recess in the top side of the base, the second recess defining a second niche configured to receive a second jaw of a second table clamp.

9. The syringe holder of claim 1,wherein:
a) the cylindrical cavity in the vertical mount has a diameter of Dl;
b) the first rounded slot in the lower barrel support has a diameter of D2; and
c) the diameter D1 of the cylindrical cavity of the vertical mount is equal in size to the diameter D2 of the first inverted rounded arch of the lower barrel support of the horizontal mount.

10. The syringe holder of claim 1, wherein the cylindrical cavity of the vertical mount has a diameter D1 that is slightly smaller than a diameter of the bottom end of the barrel of the syringe so that bottom end of the barrel of the syringe may be removably snap-fitted into the cylindrical cavity.

11. The syringe holder of claim 1, wherein the cylindrical cavity in the vertical mount has a diameter D1 that is slightly smaller than a diameter of the top end of the barrel of the syringe so that the top end of the barrel, the shoulder and the discharge orifice may be removably snap-fitted into the cylindrical cavity.

12. The syringe holder of claim 1, wherein the first rounded slot in the lower barrel support of the horizontal mount has a diameter D2 that is slightly smaller than a diameter of the bottom end of the barrel of the syringe so that the bottom end of the barrel of the syringe may be removably snap-fitted into the first rounded slot of the lower barrel support.

13. The syringe holder of claim 1, wherein the second rounded slot in the upper barrel support of the horizontal mount has a diameter D3 that is slightly smaller than a diameter of the top end of the barrel of the syringe so that the top end of the barrel of the syringe may be removably snap-fitted into the second rounded slot of the upper barrel support.

14. The syringe holder of claim 1, wherein the second inverted rounded arch of the upper barrel support of the horizontal mount is elongated to define an elongated trough configured to support the top end of the barrel of the syringe when the syringe is placed in said generally horizontal orientation on the horizontal mount.

15. The syringe holder of claim 1,wherein the base, the vertical mount and the horizontal mount are of unitary construction.

16. The syringe holder of claim 15,wherein the unitary construction comprising the base, the vertical mount and the horizontal mount is fabricated by injection molding.

17. The syringe holder of claim 15,wherein the unitary construction comprising the base, the vertical mount and the horizontal mount is fabricated by a three-dimensional (3D) printer.

18. The syringe holder of claim 15,wherein the unitary construction comprising the base, the vertical mount and the horizontal mount is fabricated from one of:
a) polyamide thermoplastic (Nylon); or
b) polyetherimide (PEI) thermoplastic; or
c) polyether ether keytone (PEEK) thermoplastic; or
d) Acrylonitrile Butadiene Styrene (ABS) thermoplastic.

19. The syringe holder of claim 1, wherein the top side of the base is substantially planar in shape.

20. The syringe holder of claim 1, wherein the bottom side of the base is substantially planar in shape.
